(19) United States
(12) Patent Application Publication
(10) Pub. No.: US 2014/0236130 A1
(43) Pub. Date: Aug. 21, 2014

(54) Catheter

(71) Applicant: John T. R. Wilson, Seattle, WA (US)

(72) Inventor: John T.R. Wilson, Seattle, WA (US)

(21) Appl. No.: 14/120,140

(22) Filed: Apr. 29, 2014

Related U.S. Application Data
(63) Continuation of application No. 13/998,397, filed on Jan. 13, 2014.

(51) Int. Cl. A61M 25/00

(52) U.S. Cl. CPC A61M 25/0043; A61M 25/0017
USPC 604/544

(57) ABSTRACT

This invention identifies cavitation as a damaging problem in catheterizing, such cavitation occurring upon removal of the catheter from the urethra. This invention shows venting the catheter tip to reduce cavitation related damage to the urinary track upon catheter removal. This invention discloses methods to reduce or prevent cavitation damage to portions of the urinary track This invention shows a vent to reduce partial vacuum formation leading to cavitation damage can be plastic “trimmer lines” used in lawn trimmers also called “weed eaters”.

### CROSS REFERENCE

This application is a continuation of prior Non-Provisional application Ser. No. 13/998,397, filed Jan. 13, 2014 entitled: “Cavitation Reduction in Catheter Removal”.

### BACKGROUND OF THE INVENTION

This invention relates to the human urinary track and to draining urine from the bladder by catheterizing. Such catheterizing sometimes causes bleeding. Certain urinary track bleeding related to catheterizing is the problem solved by this invention.

Cavitation is a hydraulic term, often used in pump design and action, meaning the formation of a partial vacuum in a liquid and the subsequent collapse of the vacuum causing cavitation damage to solid surfaces. In short term applications such as liquid rocket engine firings, a cavitating venturi may be used for regulating liquid flow without concern for cavitation damage.

In human catheterizing, bleeding is undesirable from any source in the urinary track. Sharp edges on the catheter can cause bleeding. Catheterizing without allowing sufficient time for healing of previous bleeding sources is undesirable.

Biopsies of bladder cells are done to identify bleeding sources. Biopsies have identified impact-damaged bladder cells as suspected bleeding sources.

### BRIEF SUMMARY OF THE INVENTION

The invention recognizes that damaging urinary cavitation may occur after human bladder catheterizing upon removal of the catheter and describes a method and devices to reduce or avoid that cavitation. This invention describes methods and devices to reduce or avoid cavitation between a catheter containing residual urine and a body parts interface by venting air or other fluid to the body parts interface prior to or coincident with or during removal of the catheter. By admitting the atmospheric or outside air or other fluid, no partial vacuum will be formed when the catheter is removed.

### BRIEF DESCRIPTION OF THE DRAWINGS

This invention is described using eight Figures. FIG. 1 though FIG. 6 include some enlargements for ease of presentation. The FIG. 1 and FIG. 2 depict the initial stages of catheterizing FIG. 3 and FIG. 4 depict stages of catheterizing following FIG. 2 without using this invention. FIG. 5 and FIG. 6 depict stages of catheterizing following FIG. 2 using this invention. The FIG. 1 through FIG. 6 depict cross-sections through the human body parts: urethra 2, sphincter 5, valve 6, bladder 4, left-kidney ureter 7 and right-kidney ureter 8.

FIG. 1 depicts bladder 4 being drained of urine 10 through catheter 1 with the valve 6 open. The catheter 1 is shown after being inserted through urethra 2, using lubricant 3 and pushing valve 6 to open to the bladder 4. This FIG. 1 shows prior art.

FIG. 2 depicts sphincter 5 operated to close valve 6 to interrupt the flow path from the bladder 4 to the catheter 1 thus stopping the flow of urine 10. This FIG. 2 shows prior art FIG. 2 also depicts the body parts interface region 9 between valve 6 and the catheter 1.

FIG. 3 depicts the situation following the FIG. 2 situation without using this invention. FIG. 3 depicts urine 10 and the formation of a partial vacuum 11 in the interface region 9 and elsewhere at the start of catheter 1 removal. The partial vacuum 11 extends from the body parts interface region 9, through the valve 6 and into the bladder 4.

FIG. 4 depicts the situation that follows the FIG. 3 situation without using this invention. FIG. 4 depicts the collapse of the partial vacuum 11 into pieces and the impacting of urine 10 onto solid surfaces of bladder 4, valve 6 and ureters 7 and 8.

FIG. 5 depicts the situation following the FIG. 2 situation using this invention. FIG. 5 also depicts this invention with the introduction of vent 12 into the flow path of catheter 1 to near the tip of catheter 1 in the body parts interface region 9. The vent 12 is a flexible plastic line. FIG. 5 also depicts the sphincter 5 operated to close valve 6 to interrupt the flow path from the bladder 4 to the catheter 1 thus stopping the flow of urine. FIG. 5 also depicts the introduction of atmospheric air 13 through the introduction of vent 12.

FIG. 6 depicts the situation following the FIG. 5 situation using this invention. FIG. 6 also depicts the catheter 1 partially removed from the body parts interface region 9 with no partial vacuum formation, nor vacuum collapsing, nor urine 10 impacting solid surfaces of bladder 4, valve 6, urethra 2, or ureters 7 and 8.

FIG. 7 depicts a catheter 1 in approximately full size. The tip of the catheter 1 is to the left and has the rounded point. The tip is inserted in the urethra 2 in catheterizing, and in FIG. 7 the tip has the elongated opening outline, which is the entry for urine from the bladder 4. The outlet of urine from the catheter during catheterizing has the squared end.

FIG. 8 depicts a vent 4 in approximately full size.

### DETAILED DESCRIPTION OF THE INVENTION

The invention will be described by describing the drawings in more detail in a catheterizing sequence.

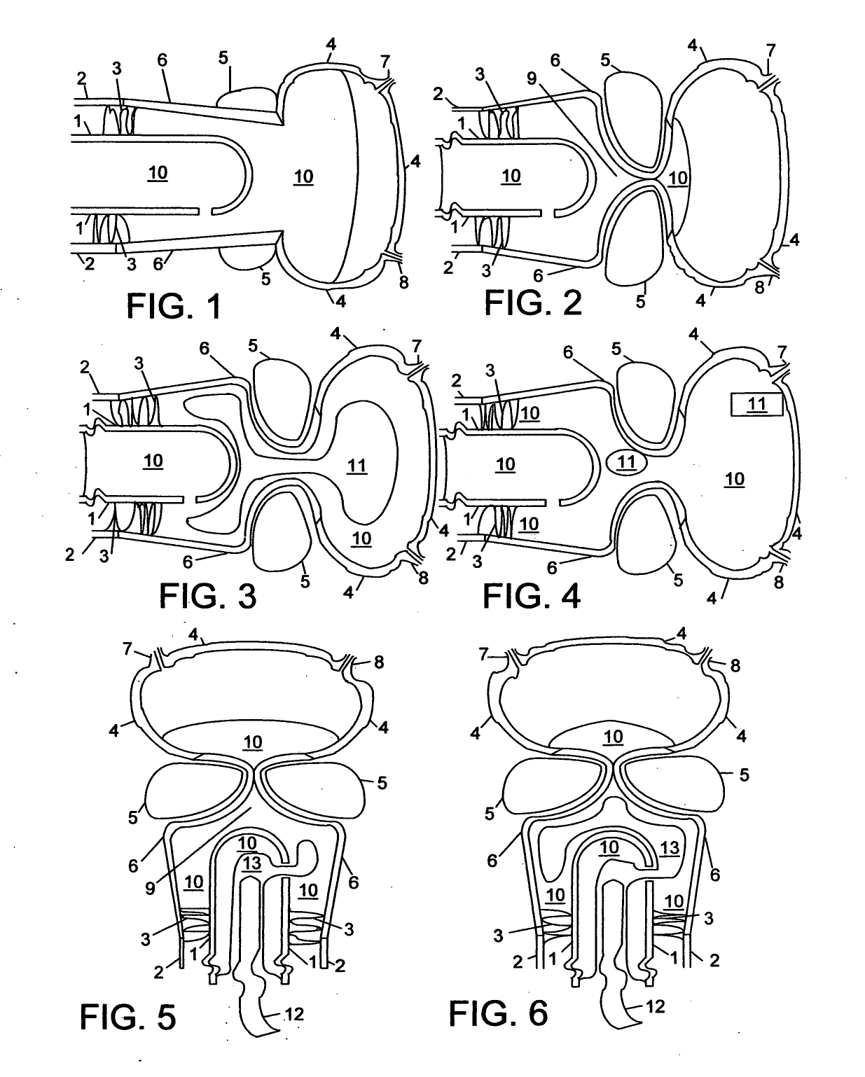

In FIG. 1 is depicted catheterizing in the process of draining urine 10 from the bladder 4 into the catheter 1 via the hole in catheter 1 to the outlet of catheter 1. Catheterizing starts when the catheter 1 and the mouth of urethra 2 have been coated with lubricant 3 and the catheter 1 has been inserted up the urethra 2 to push open the valve 6 and start flow of urine 10.

In FIG. 2 is depicted catheterizing in which the draining of urine 10 from bladder 4 has been determined to be completed and the sphincter 5 has constricted and closed valve 6. A small amount of urine 10 remains in bladder 4 and some urine 10 remains in the tip of catheter 1 and for some distance to the left in catheter 1.

The FIG. 3 depicts the situation of catheter removal without the use of subject invention following from the FIG. 2 situation. The FIG. 3 shows catheter 1 slightly withdrawn to the left causing the formation of the partial vacuum 11 in body parts interface region 9, previously shown in FIG. 2, and the partial vacuum 11 permeates the in urine 10

The FIG. 4 depicts the situation following the FIG. 3 situation which includes the expansion of the partial vacuum 11 through valve 6 and into bladder 4. The partial vacuum 11 collapses into pieces, depicted as rectangular and spherical, next to valve 6, bladder 4 and ureters 7 and 8. The urine 10 permeates the partial vacuum 11 pieces and as those vacuum pieces collapse the urine 10 makes damaging impacts. This is called cavitation. This is what this invention prevents.

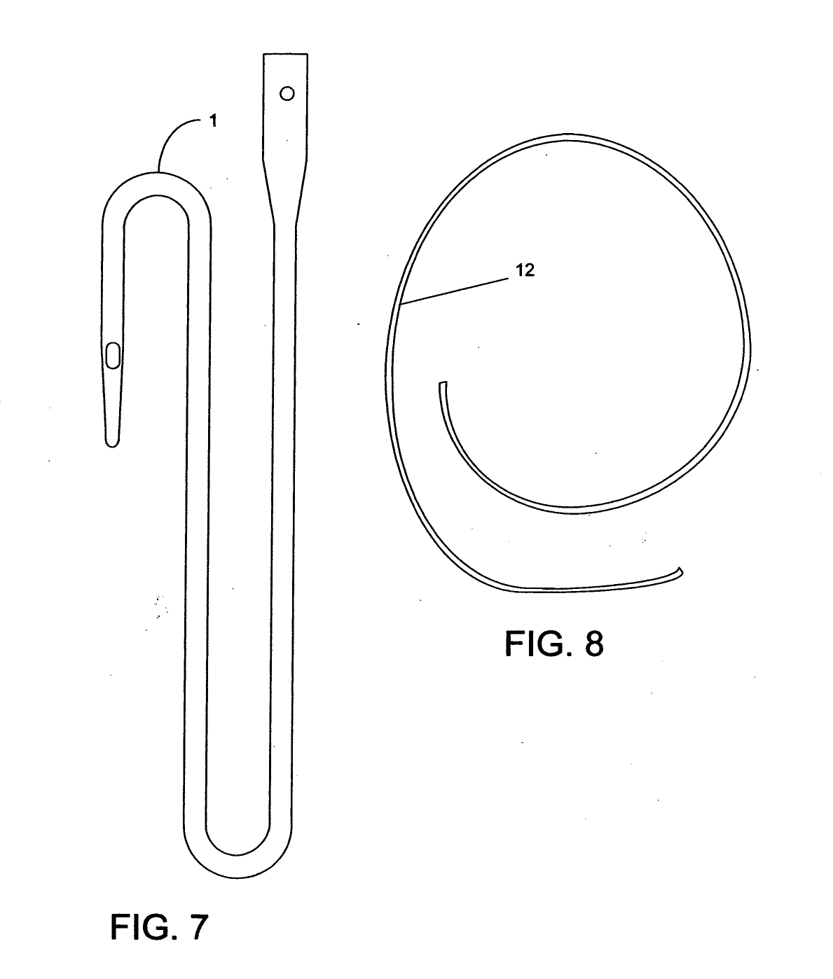

In FIG. 5 is depicted the situation following from the FIG. 2 situation using this invention, in which the vent 12 of this invention is introduced into the catheter 1. The vent 12 is a flexible plastic line which may be a tube. This vent 12 is introduced into the discharge end of catheter 1, shown as the squared end in FIG. 7, and pushed up to the tip of catheter 1, so it is placed as shown in FIG. 5, in the body parts interface region 9. The stiffness of vent 12 is enough to allow the vent 12 to be pushed up the catheter 1. By this action the atmospheric air 13 is introduced into the body parts interface region 9. The vent 12 used is a flexible plastic line about an inch longer than the catheter 1 for ease of manual manipulation and of a diameter to fit within the catheter 1 flow path. The ends of the vent 12 are filed to prevent gouging the inside diameter of the catheter 1.

The FIG. 6 depicts the situation following from the FIG. 5 situation using this invention. The FIG. 6 shows the catheter 1 slightly withdrawn downward causing a vacancy in the urine 10 in the body parts interface region 9 which vacancy is filled by atmospheric air 13; at this catheter withdrawal there is no partial vacuum 11 formed, and there is no cavitation nor the associated damage.

From the situation depicted in FIG. 4, to complete a catheterizing, the catheter 1 would be fully withdrawn from the urethra 2.

From the situation depicted in FIG. 6, to complete a catheterizing, the catheter 1 and vent 12 would be fully withdrawn from the urethra 2.

A working model of this invention has been made and tested. The catheter 1 has been a Bard 16 Fr. coude tip, 16 inches long. The vent 12; has been an Arnold Trimmer Line 0.065 inches diameter; the ends have been filed to prevent gouging the inside diameter of the catheter.

### Future Catheters

A catheter of the future may include an atmospheric air vent which would be controlled to connect the body-parts-to-catheter-tip interface to atmospheric air prior to removing the catheter. This control could be a motion detector for the catheter removal, a permeability or anti-permeability membrane between atmospheric air and the mine liquid or some other control.

While certain preferred embodiments of the present invention have been disclosed in detail, it is to be understood that various modifications in its structure may be adopted without departing from the spirit of the invention or the scope of the following claims.

I claim as my invention:

1. A cavitation reduction operative between a catheter and a body parts interface region when the catheter is removed from the interface region to reduce &image to the body parts.

2. A cavitation reduction as in claim 1 comprising a fluid vent inserted in the catheter outlet to the interface region.

3. A cavitation reduction as in claim 2 wherein the fluid vent comprises a flexible plastic line.

4. A cavitation reduction as in claim 1 wherein the catheter comprises a fluid vent.

5. A cavitation reduction as in claim 4 wherein the catheter comprises a liquid-soluble block of the vent.

6. A cavitation reduction as in claim 4 wherein the liquid-soluble block comprises a urine-soluble block.

7. A cavitation reduction as in claim 3 wherein the flexible plastic line comprises a trimmer line.

8. A cavitation reduction as in claim 1 wherein the catheter also comprises a controlled fluid vent of the body parts interface region coincident with or prior to removal of the catheter.

9. A cavitation reduction as in claim 8 wherein the controlled fluid vent comprises a membrane permeable to atmospheric air but not permeable to liquid.

10. A cavitation reduction as in claim 8 wherein the controlled vent comprises a motion detector for catheter removal.

11. A cavitation reduction as in claim 1 wherein the catheter also comprises a controlled fluid vent controlled to vent the body parts interface region during removal of the catheter.

12. A method of catheterizing comprising venting air or other fluid to a body parts region prior to or coincident with removal of the catheter to prevent or reduce cavitation.

13. A method of catheterizing comprising a fluid vent of the catheter tip prior to or coincident with catheter removal to prevent a partial vacuum formation at the catheter tip.
